(19) United States
(12) Patent Application Publication
(10) Pub. No.: US 2012/0238887 A1
(43) Pub. Date: Sep. 20, 2012

(54) HYDROSTATIC FINGER CUFF FOR BLOOD WAVE FORM ANALYSIS AND DIAGNOSTIC SUPPORT

(76) Inventors: David W. Gerdt, Faber, VA (US); Charles Adkins, Earlysville, VA (US); Martin C. Baruch, Charlottesville, VA (US)

(21) Appl. No.: 13/231,703

(22) Filed: Sep. 13, 2011

Related U.S. Application Data
(63) Continuation-in-part of application No. 12/854,954, filed on Aug. 12, 2010, now abandoned.
Application No. 12/854,954 is a continuation-in-part of application No. 12/537,228, filed on Aug. 6, 2009.
Application No. 12/537,228 is a continuation-in-part of application No. 11/500,558, filed on Aug. 8, 2006, now Pat. No. 8,100,835.
Application No. 11/500,558 is a continuation-in-part of application No. 10/502,932, filed on Jul. 29, 2004, now Pat. No. 7,087,025.
Application No. 10/502,932 is a continuation-in-part of application No. 09/763,657, filed on Feb. 26, 2001, now Pat. No. 6,723,054.

(51) Int. Cl. A61B 5/022; A61B 5/026; A61B 5/025

(52) U.S. Cl. 600/499; 600/500; 600/504

(57) ABSTRACT

A hydrostatic finger cuff for blood flow property analysis is provided which includes an elongated substrate member having a pair of opposing long edges and a pair of opposing short edges. The hydrostatic finger cuff is configured to form a frustoconical shell when the ends of the cuff are overlapped and releasably connected together. The interior of the frustoconical shell conforms to the shape of the finger or thumb. One side of the elongated member has an inflatable member that has a pressurizable interior region. A tube is fixed to the inflatable member and is in pneumatic communication with the interior of the inflatable member inflatable to a maximum of no more than 60 mmHg. The inflatable member completely circumscribes the finger and provides substantially uniform contact across the entire length of a phalange.

### CROSS REFERENCE TO PRIOR APPLICATION

This application is a continuation-in-part of co-pending U.S. patent application Ser. No. 12/854,954 for Hydrostatic Finger Cuff Blood Wave Form Analysis filed Aug. 12, 2010; Blood Pressure Determination Based on Delay Times between Points on a Heartbeat Pulse, pending patent application Ser. No. 12/537,228 filed Aug. 6, 2009; Method for Arterial Pulse Decomposition Analysis for vital Signs Determination, pending patent Ser. No. 11/500,558 filed Aug. 8, 2006, which is a C-I-P of U.S. Pat. No. 7,087,025 for Blood Pressure Determination Based on Delay Times Between Points on a Heartbeat Pulse issued Aug. 8, 2006, and Wrist Plethysmograph, Ser. No. 11/803,643 filed May 15, 2007, and Apparatus and Method for Measuring Pulse Transit Time, U.S. Pat. No. 6,723,054 issued Feb. 26, 2001, all of which are incorporated herein by reference, as though recited in full.

### GOVERNMENT INTEREST STATEMENT

The ONR (Office of Naval Research) contract N00014-10-C-0204

### BACKGROUND

1. Field of the Invention

The present invention relates generally to a system for measuring an arterial pulse, and more particularly to a means by which arterial pulse wave form can be continuously monitored with a noninvasive device that makes direct mechanical contact with the user's finger but without occluding blood flow in the finger.

2. Background of the Invention

The pressure pulse, the mechanical representation of the blood flowing in the artery, is generally believed to be best detected at the classic pressure points that are well known and whose locations are widely published in the literature. At these points the artery is close to the surface of the skin so that with application of light constrictive pressure (palpation), the pulsations caused by the heartbeat can be sensed mechanically as pulsations in the constrictive force.

### SUMMARY OF THE INVENTION

In accordance with an embodiment of the invention, a hydrostatic finger cuff for arterial pulse property analysis is provided which includes an elongated substrate member which has a pair of opposing long edges and a pair of opposing short edges. The hydrostatic finger cuff is configured to form a frustoconical shell when the ends of the cuff are overlapped and releasably connected together. The interior of the frustoconical shell conforms to the shape of the middle phalange of a finger or the first phalange of the thumb. If a finger is used it is preferably the middle finder, in the region from the distal joint to the proximal joint. The finger cuff is used by one patient and is disposed of after being used by one patient with the intension of preventing nosocomial infections. The finger cuff may be disposed of after a period of use by a patient and replaced with a new, unused finger cuff for further use by the patient.

In a preferred embodiment the pair of opposing long edges has a radius of curvature in the range from about 7 inches to about 13 inches. In a further preferred embodiment, the first of the long side edges has a radius of curvature in the range from 10 inches to about 13 inches and a second of the long side edges has a radius of curvature in the range from 10 inches to about 7 inches. In a more preferred embodiment the first of the long side edges has a radius of curvature in the range from 10 inches about 12 inches and a second of the long side edges has a radius of curvature in the range from 10 inches to about 8 inches.

In another embodiment of the invention an inflatable member mounted on the obverse side of the elongated member and the inflatable member has a pressurizable interior region. A tube is fixed to the inflatable member and is in pneumatic communication with the interior of the inflatable member. The inflatable member is preferably a urethane membrane peripherally sealed to a urethane substrate member. One part of a two part connector, preferable one of a hook and loop member is affixed to the obverse side of the elongated member and positioned proximate a first side edge of the elongated substrate member. The inflatable member is positioned proximate a second side edge of the elongated member, and the other of a hook and loop member is affixed to the reverse side of the elongated member in the region of the inflatable member. Preferably, the loop member is proximate the inflatable member and the hook member is distal the inflatable member, thus optimizing the ability of the cuff to conform to the contour of the user's finger when the cuff is wrapped around the finger. Alternatively, a releasable, reusable adhesive can be used in place of the hook and loop connector. Detachable, reusable pressure sensitive adhesives (PSAs) based on polyurethane are described in, for example, U.S. Pat. Nos. 6,040,028 and 5,102,714, and Japanese patents, JP 08 188 755 and JP 06 279 741. The PSA provide the advantage of enabling better conformation of the cuff to the curvature of the finger.

In a preferred embodiment of the invention, the substrate member, the inflatable member, and the tube are formed from polyurethane.

In a further embodiment of the invention, the long side edges of the elongated substrate member have a length in the range from 3 inches about 6 inches.

In a further embodiment of the invention a first of the long side edges of an elongated substrate member has a radius of curvature in the range from 10 inches about 12 inches and a second of the long side edges has a radius of curvature in the range from 10 inches about 8 inches.

In a still further embodiment of the invention the inflatable member is substantially rectangular and has an active surface area in the range from 0.9 to 2.5 square inches.

In another embodiment of the invention the inflatable member is substantially rectangular and has an active surface area of at least 1.5 square inches, and preferably, an active surface area in the range from about 1.5 to 2.5 square inches in order to accommodate a large finger.

In a further embodiment of the invention the inflatable member is substantially rectangular and has an active surface area in the range from about 0.9 to 1.3 square inches. The first side of the active area of the inflatable member has a width that is slightly less that the distance between the distal joint of a finger and the proximal joint of the finger, and preferably extends from proximate the distal joint of a finger to a point proximate the proximal joint of the finger. Most preferably the length of the first side is in the range from 0.6 inches to 1.2 inches in order to accommodate a small finger and in the range from 1.25 inches to 0.88 inches to accommodate a large finger. Preferably, the finger is the user's middle finger.

In a further embodiment of the invention the inflatable member is substantially rectangular and has a length to width ration in the range from 1.5 to 1, to 2.5 to one, preferably it has a length to width ration in the range from 1.75 to 1 to 2.25 to 1, and most preferably, about 2 to 1.

In a further embodiment of the invention the active area of the inflatable member has an edge whose length is in the range from 0.63 inches to 0.88 inches and a second side or edge whose length is in the range from 1 inch to 1.5 inches.

In another embodiment of the invention a first side of the active area of the inflatable member has a length in the range from 1.25 inches to 0.88 inches and a second side of the active area of the inflatable member has a length in the range from 2.25 inches to 1.5 inches. With respect to the average circumference of the user's middle phalange, the inflatable member is preferably long enough to cover at least ½ and most preferably, at least ⅔ of the circumference of the middle flange of the finger, with the middle finger being the preferred finger.

In a further embodiment of the invention the tube has an inside diameter in the range from 0.05 to 0.075 inches in order to provide the preferred level of fluid communication between the inflatable member and a pressure sensor.

It is noted that dimensions represent one of the critical features of the invention in that the inflatable member must be completely circumscribe the finger and provide substantially uniform contact across the entire length of a phalange, in particular, the middle phalange of the middle finger. This is in contrast with optical sensors and palpating devices which in essence, focus on the region of the finger's artery.

In another embodiment of the invention a hydrostatic finger cuff for blood flow property analysis, comprises an elongated substrate member, an inflatable member mounted on the obverse side of the elongated member, a tube fixed to the inflatable member and in pneumatic communication with the interior of the inflatable member, one of a hook and loop member affixed to the obverse side of the elongated member and positioned proximate a first side edge of the elongated substrate member, and a pressure relief member. The pressure relief member is in fluid communication with the inflatable member, and is set to open and release pressure at a pressure level no higher than 1.2 psi.

In still another embodiment of the invention the tube has a terminal end proximate the inflatable member and a terminal end distal the inflatable member, and the pressure relief member is a poppet valve affixed to the tube at a point between the proximal terminal end and the distal terminal end and is set to open and release pressure at a pressure level of up to 1.2 psi and most preferably at no greater than 1.1 psi. The poppet valve can be attached to the tube by a “Y” or “T” connector.

In another embodiment of the invention the pressure relief member is a frangible member affixed to the substrate member and in fluid communication with the interior of the inflatable member. The frangible member can be a membrane that is designed to open and release pressure at a pressure level of up to 1.2 psi. and preferably at a pressure no greater than 1.1 psi.

In a further embodiment of the invention blood flow properties are analyzed and monitored with a finger cuff device which comprises an elongated substrate member, having an obverse side and a reverse side, an inflatable member mounted on the obverse side of the elongated member, the inflatable member having a pressurizable interior region, a tube fixed to the inflatable member and in pneumatic communication with the interior of the inflatable member, one side of a connector member is affixed to the obverse side of the elongated member and positioned proximate a first side edge of the elongated substrate member, the inflatable member is positioned proximate a second side edge of the elongated member, and the other of part of the connector member is affixed to the reverse side of the elongated member. The monitoring process comprises the steps of:

a—encircling a user's finger with the finger cuff,
b—affixed the two parts of the connector member together such that a cylindrical cuff is formed around a middle phalange of a finger and the inflatable member is in circumferential contact with the finger of the user,
c—inflating the inflatable member to a pressure below the user's diastolic pressure, and
d—generating pressure fluctuations which correspond to the user's blood pressure.

Preferably the inflatable member is inflated to a pressure in the range from 30 mmHg to 60 mmHg and most preferably to a pressure in the range from 40 mmHg to 50 mmHg.

In a further embodiment of the invention pressure pulses are transmitting from the inflatable member to a pressure sensor, and an analog output from the pressure sensor is converted to a digital signal. The digital signal to a computer where the digital signal is converted to a pulse wave form and the pulse wave form is converted to blood pressure values.

### BRIEF DESCRIPTION OF THE DRAWINGS

FIG. 1 is a side view of the unwrapped finger cuff in accordance with the present invention;

FIG. 2 is a side view of the wrapped finger cuff in accordance with the present invention;

FIG. 3 is a side view of an alternate embodiment of an unwrapped finger cuff in accordance with the present invention;

FIG. 4 is a face view of the unwrapped finger cuff in accordance with the present invention;

FIG. 5 is a face view of an additional embodiment unwrapped finger cuff in accordance with the present invention;

FIG. 6 is a dorsal perspective view of the finger cuff on a user's hand in accordance with the present invention;

FIG. 7 is a volar perspective view of the wrapped finger cuff illustrating the conical configuration in accordance with the present invention;

FIG. 8 is a perspective view of the finger cuff in its conical form, in accordance with the present invention;

FIG. 9 is a volar perspective view of the wrapped finger cuff placed on the first phalange of the user's thumb in accordance with the present invention;

FIG. 10 is a plan view of an inflatable membrane and tube in accordance with an embodiment in accordance with the present invention;

FIG. 11 is a plan view of an alternate embodiment of the invention in accordance with the present invention; and

FIG. 12 is a plan view of an alternate inflatable membrane and tube in accordance with another embodiment of the present invention.

### DESCRIPTION OF EMBODIMENTS OF THE INVENTION

### Definitions

For the purposes of the present invention, the term “piezoelectricity” refers to the ability of crystals and certain ceramic materials and certain polymers to generate a current and voltage in response to applied mechanical stress. The piezoelectric effect is reversible in that piezoelectric crystals, when subjected to an externally applied voltage, can change shape by a small amount. (For instance, the deformation is about 0.1% of the original dimension in piezo element.) The effect finds useful applications such as the production and detection of sound, generation of high voltages, electronic frequency generation, microbalance, and ultra-fine focusing of optical assemblies.

For the purposes of the present invention, the term “piezo element” refers to any material that has capability of generating piezoelectricity.

For the purposes of the present invention, the term “plethysmograph” refers to an instrument that measures variations in the size of an organ or body part on the basis of the amount of blood passing through either as blood flow (about 2 cm/sec) or pulse propagation velocity (5-15 m/sec) or present in the part.

For the purposes of the present invention, the term “PZT” refers to a piezo element, as for example, one of lead zirconate titanate, a material that shows a marked piezoelectric effect as well as any other electroceramic that contains the properties necessary achieve the results set forth herein.

For the purposes of the present invention, the term “transimpedance amplifier” refers to a circuit for converting current input into voltage output. A typical situation is the measuring of current using a voltmeter to measure the resistive drop or IR drop across a known resistor. A current-to-voltage converter is a circuit that produces voltage preferably linearly in an increasing amount in response to an increasing current. The converter acts as a linear circuit with transfer ratio k=V_{OUT}/I_{IN} [V/A] having dimension of resistance. The active version of the circuit is also referred to as a transresistance or transimpedance amplifier.

For the purposes of the present invention, the term “light coupling”, as employed herein, refers to a minimal coupling level. The coupling is sufficient to provide a firm contact between the cuff and the finger, but producing no more than minimal interference with the flow of blood in the artery of the finger.

For the purposes of the present invention, the term “firm contact” as employed herein refers to a sufficient contact between the user's finger and the bladder to generate an analogue signal that corresponds to the user's blood pressure. By way of contrast, U.S. Pat. No. 4,726,382 discloses a finger cuff in which “[o]uter cuff label 18 is sufficiently unstretchable so as to allow inflation of the inflatable bladder 12 to affect circulation of blood within the arterial system of the patient's finger”. (See column 4, lines 51-59)

For the purposes of the present invention, the term “rectangular” as employed herein refers to any generally rectangular shape inclusive of a rectangle has chamfered corners, or filleted corners as in what is commonly referred to as “race track” shape.

For the purposes of the present invention, the term “finger”, as employed herein, refers to all of the five digits of the mammalian hand. Specific reference to particular digits is made where referencing optimum usage however generic use of the term finger can indicate optimum and non-optimum placement of the finger cuff.

The present invention relates to wireless and noninvasive physiological monitoring system in the form of a hydrostatic finger cuff for measuring heart rate variability (HRV), blood pressure, hypovolemia, hypervolemia, inter-beat interval, abnormal patterns, arrhythmia, and other physiological cycles. Monitored data can be stored within the device for later download using a connection to a PDA or PC, via a connection such as USB, Bluetooth, etc. Alternatively the data can be sent to the receiving device in real time.

The incorporated relative pressure sensor must have sufficient sensitivity that little deformation is necessary to couple the sensor to the pulse, thereby enabling the device to be used for many hours, or even days, on a continuous basis.

A standard, commercially available pressure sensor, such as a relative (gauge) or absolute sensor, can be used to keep the pressure in the cuff constant and is no different from a manometer. Essentially, it is used as a gauge pressure sensor since it is open to the atmosphere on the other side. However, as the sole gauge, it does a poor job of measuring changes in pressure unless they are very, very slow, like atmospheric pressure.

A second pressure sensor is used to measure the time rate of change of pressure. It will not measure static pressure, either absolute or gauge. Piezo disks mounted in a ring have been used to measure the pulse pressure wave. They were shoved up against the radial artery and produced a wave form of the arterial pulse. The problem with these was that minor motion caused the loss of subsequent beats for maybe six to ten seconds. It is possible to make them settle very quickly, but the components become very large and not amenable to small devices. It has been found that by using a transimpedance amplifier the settling time can be reduced to less than one heart beat cycle with tiny components. This action produces the time derivative of the pulse pressure pulse.

The pulse decomposition analysis principle is used to analyze the arterial pressure pulse. The finger cuff provides continuous pressure readings by de-convolving the pulse waveform into its constituent component pulses by a process known as Pulse Decomposition Analysis (PDA). For a full disclosure of PDA technology see U.S. Pat. No. 7,087,025, Blood Pressure Determination Based on Delay Times between Points on a Heartbeat Pulse, pending patent application Ser. No. 12/537,228, Detection of Progressive Central Hypovolemia, filed Aug. 6, 2009, pending patent: Ser. No. 11/500,558, Method for Arterial Pulse Decomposition Analysis for vital Signs Determination, and Diagnostic Support Apparatus, PCT/US10/43914, filed Jul. 30, 2010, which are incorporated herein by reference, as though recited in full.

In prior art optical systems, it is the blood oxygen saturation that is measured. When this is done, a waveform is generated that shows how the oxygenation changes during a pulse cycle. The optically derived waveform looks something like a pulse pressure waveform, but it is not, and it cannot be used to obtain BP via pulse decomposition analysis.

The design of the finger cuff of the present invention enables the device to be used without significantly affecting the flow of blood because the inflation of the cuff need only be sufficient to produce a light coupling to the arteries of the finger. Ideally, the finger/pressure cuff does not change the inner diameter of the artery at all and therefore does not affect the flow of blood. The pressure in the cuff is preferably less than the diastolic pressure in the artery, and preferably, no greater than about 50 mmHg The lower limit of the pressure is sufficiently high to enable the light coupling with the artery but sufficiently low that the interference with the blood flow, if any, is minimal. Minimal interference means that the cuff can be used for extended periods of time, that is, for multiple hours or days.

The concept of externally loading an artery needs to be understood to understand why the finger cuff works in an entirely different way than the radial bladder. An easily visualized example is to think of a balloon at say 10 PSI. The skin of this balloon is under elastic tension and any disturbance such as a pulse inside the balloon will stress the skin due to this disturbance. During the disturbance or pulse, the balloon will momentarily store more than just the background pressure of 10 PSI. Now put this balloon into another balloon that is at atmospheric pressure, or effectively zero PSI. Nothing changes. However, if the outside of the second balloon is pressurized until it is ten PSI, then everything inside of the second balloon is at 10 PSI. There is no elastic tension in the first balloon and pulses inside the first balloon will modulate the elastic tension of the second balloon skin. This action is called unloading the artery (the first balloon).

The finger cuff is wrapped around the measurement site and inflated to a low pressure near, for example, to 50 mmHg to increase the contact pressure. The pulse then causes a small variation in the internal pressure in the cuff due to a very small volume change as the blood surges past the site.

In order to avoid occluding blood flow, the pressure is maintained below the diastolic pressure and is preferably in the range from 30 to 70 mmHg and most preferably in the range from about 35 to 55 mmHg. The pressure is determined on the basis of maintaining good contact with the finger, or more specifically, the pulse wave. The cuff surrounds the phalange and applies uniform circumferential pressure. Maintaining a constant or consistent pressure is not necessary but it is essential to set a maximum level for the pressure so as to avoid occluding the blood flow. However, a minimum pressure is critical from the standpoint of maintaining good contact with the finger in order to sense the pulse pressure wave.

A piezoelectric buzzer element is attached across the inputs and the gain (Transimpedance) is varied a little to adjust the signal level to fit the finger cuff. The bandwidth of the device is adjusted slightly to remove sensitivity to outside noise sources. The signal bandwidth contains the fundamental at about 1 Hz and the signal features which extend to about 60 Hz.

A pneumatic coupling is provided between the finger cuff and the analog signal generator. Although the finger cuff will work on any finger, the optimum site for the finger cuff has been found to be the first phalange of the thumb. Extremely accurate results can also be obtained using the middle phalange of a finger, preferably the middle finger.

The thumb provides optimal results since, as known in the medical arts, the thumb contains the princeps pollicis artery which arises from the radial artery. Therefore a signal from the thumb is almost directly a signal from the radial artery where most of the previous attempts to do continuous BP have and now occur, such as in applanation tonometry.

The other fingers have the proper palmer digital arteries and arise from the ulnar artery. The mean diameter of the radial artery is 28% larger than the ulnar artery (Riekkinen H V, Karkola K O, Kankainen A. The radial artery is larger than the ulnar. Ann Thorac Surg 2003; 75:882-4). This means the blood flowing to the thumb artery is greater that he blood flowing to the other digits. In addition, the radial artery serves only a single digit, while the ulnar artery serves four digits.

The use of the thumb has now been found to be advantageous because temperature appears to have a relationship to the functioning of the finger cuff device. Thus, the use of the thumb is advantageous because the size of the artery also directly relates to temperature. It is well known that Raynaud's disease, low thyroid levels, anemia, diabetes, heart disease, cancer, arthritis, carpal tunnel, tendonitis and many other medical conditions can produce cold hands. That is because extreme vasoconstriction of the blood vessels decreases blood flow, and cools the fingers. In thermography of a patient with such a problem, the thumb can be seen to typically have a higher temperature than the other fingers. As the disclosed finger cuff will be used primarily on patients who are injured or ill, and there is a lack of blood flow as well known in bedridden patients. (See http://www.handresearch.com/news/cold-hands-warm-heart-raynaud-poor-circulation-fingers.htm)

Further, the thumb bladder, although the same as the finger bladder does not suffer the mechanical noise problems that arise from the carpal tunnel. Almost any movement of any part of the arm causes some rubbing between taught tendons inside the tunnel producing low frequency noise. This low frequency clatter has spectral power in the same band as the heartbeat pulse, about 0.01 to 30 Hz, abounds and cannot be always completely filtered out.

The analog signal generator is preferable a piezo element, although other elements can be used that produce the same result, mounted in a pressure housing which includes a pressure pump and can include an analog to digital converter, a transimpedance amplifier, a data storage member, and a signal transmitter. The data storage member can include a solid-state memory device. The various physical, electrical, and electronic components are well known in the art and are not narrowly critical.

With the piezo element mounted around its edge so that it is pressure tight but free to move, the applied pressure causes the element to bend like a drumhead. The bending causes a charge to develop on the outer surface of the piezo material that is proportional to the pressure. Measurement of the charge is thus a measure of the pressure. With sensitive equipment, that measurement gives the pulse signature that is used for analysis.

There are various methods for measurement of the charge, though the differences can be significant. A voltage amplifier will measure the total pressure on the piezo. The voltage change due to the pulse is small compared to the total pressure change on the sensing element, and the amplifier must accommodate the full pressure change that occurs. If enough amplification is used to see the pulse signal and its features, the signal runs into the power supply rails most of the time and is not useable. This, however, requires the patient to be very still and the attendant to be very careful to produce a useable signal.

A preferred method essentially shorts the surfaces of the piezo and measures the current produced as the charge migrates due to the change in pressure. The amplifier, in this case, is called a Transimpedance amplifier because it produces a voltage change proportional to the current at its input. (Impedance is Volts/Current.) Using this configuration, the voltage output is only present as the pressure changes. The output stays centered around zero volts and significant amplification can be applied which only affects the signal.

### Finger Cuff Design

The finger cuff departs from prior art devices in that it does not attempt to palpate the finger. Pressure is not applied to the finger artery but rather, the finger is ringed by a cuff which circumferentially applies pressure to the finger, that is, it squeezes the finger.

The finger cuff is entirely constructed from polyurethane. Polyurethane film (two thousands of an inch thick) that is bonded to a 5-7 mils polyurethane outer layer of hook and loop material, preferably in a one-step operation using radio frequency welding. This helps to make the cuffs inexpensive because there is little labor component needed in construction. Preferably, the outer layer to which the inner layer is bonded, is the loop section of the hook and loop material.

Other materials, in particular polymeric material currently available or hereinafter developed, can be used provided they do not hyperextend and meet the other criteria set forth herein. It should be noted that at present, it is very difficult or impossible to bond polyurethane to other polymeric materials such as silicone or polyethylene.

Although polyurethane film is known of as a very skin friendly material and used for disposable upper arm cuffs in sphygmomanometers as well as sometimes used as disposable sheets in hospitals, is not used frequently for hook and loop connectors. The polyurethane hook and loop system allows only for about a hundred make and break uses before the system becomes weak and undependable. By way of contrast, hook and loop materials other than polyurethane can last for thousands of make and break connections and these are found on garments, and in similar applications.

The use of an adjustable connector, such as hook and loop material, provides the necessary gross connection between the bladder of the disclosed cuff and the user's finger. The expansion of the bladder is not used to accommodate a loose fitted cuff but rather to make a minor adjustment to achieve a firm contact between the bladder and the user's finger.

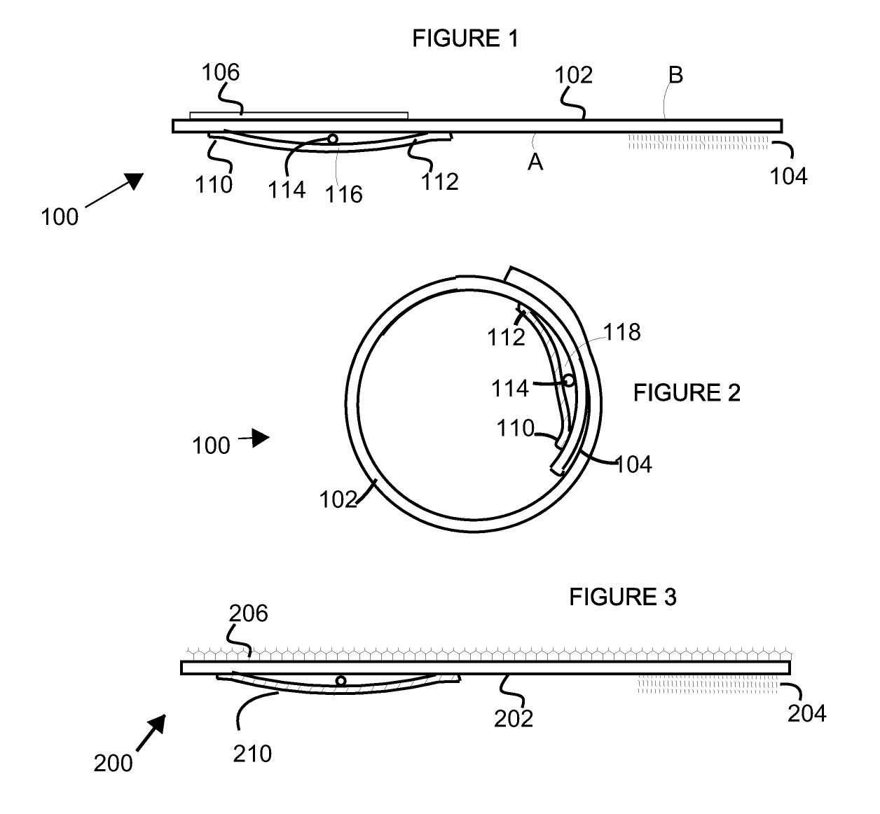

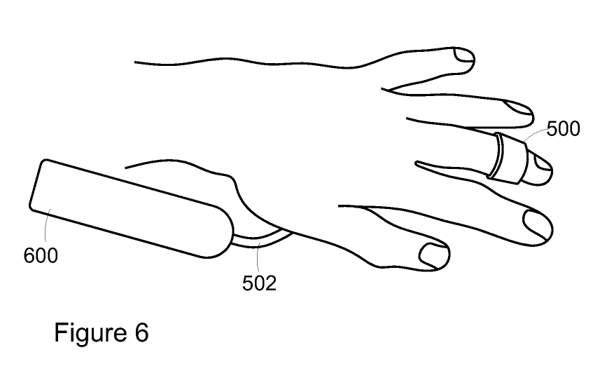

The embodiment illustrated in FIGS. 1 and 2 shows a finger cuff 100 having a hook section 104 affixed to interior side A and a loop section 106 affixed to exterior side B. The hook section 104 and the loop section 106 are affixed not only at opposite sides but also at either end of the substrate member 102 and are used to secure the cuff 100. An inflatable sensing member 110 is positioned on the interior side A of the substrate 102 approximately opposite the loop section 106 and contacts the user's finger. In these embodiments, the sensing member 110 consists of an inflatable membrane 116 and tube 114. The periphery of the inflatable membrane 116 is fused to the substrate member 102, as indicated at edges 112 and 113. The inflatable member 116 is configured to enable the sealed interior region 118 formed by the membrane 116 and the substrate 102, to be pressurized to form, in conjunction with the tube 114, the pressure sensor 110. The tube 114 is in pneumatic communication with the interior region 118 and the electronic components housing 600 of FIG. 6.

The tube 114 is preferably urethane with an interior diameter of about one-sixteenth (0.0625) inch and an exterior diameter of about one eight (0.125) inch.

In the embodiment illustrated in FIG. 3, the cuff 200 uses a sensing member 210 constructed the same as the sensing unit 110 of FIGS. 1 and 2. In this embodiment, however, the loop section 206 extends along the entire length of the substrate 202. The hook section 204 preferably is only at the end of the substrate 202 to prevent the hook section 204 from coming into contact with the user's skin and causing discomfort.

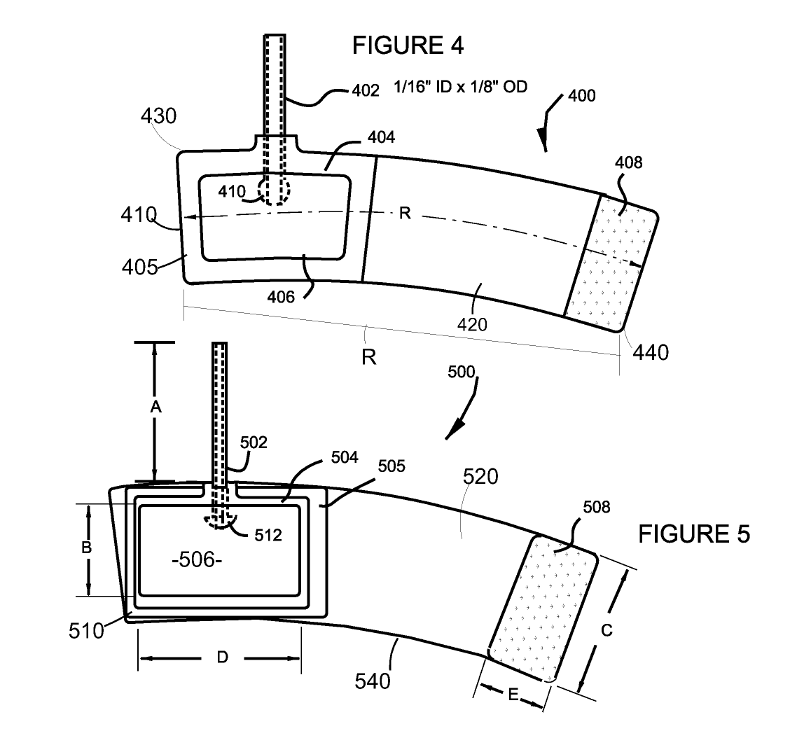

In FIGS. 4 and 5 the face of two embodiments of the cuff disclosed herein are illustrated. In FIG. 4, the cuff 400 has the hook material 408 at one end of the substrate 420 and the sensing member 410 at the opposite end. The inflatable membrane 405 is shown with the inflatable portion 406 sealed along its periphery 404. The tube 402, which extends from the inflated portion 406 to the receiving electronics (not illustrated), is maintained in place through the seal between the substrate 420 and the inflatable membrane 405. The tube 402 can also be provided with a lock 410 that provides a back up to prevent the tube 402 from being accidentally removed from the inflated portion 406. The tube 402 is sealed between the substrate 420 and the inflatable membrane 405 at the peripheral region 404, and the lock 410 is another of the safety redundancies provided within the system. The lock 410 serves to resist the tube being pulled out of the inflatable region 406 formed by the substrate 420 and the inflatable membrane 405.

As illustrated clearly in these figures, the substrate component of the finger cuff is preferably not rectangular but rather has two opposing curved edges 430 and 440, whose radius of curvature “R” is in the range from about 13 inches to about 7 inches.

In FIG. 5, an alternate embodiment, as well as preferred proportions, is illustrated. As with previous embodiments, the substrate 520 has the hook portion 508 at the end opposite that of the sensing member 510. The sealed portion 504 is at the periphery of the inflatable membrane 505. It is highly preferable, that the sealed portion 504 securely seals the tube 502 that extends into the interior of the inflatable membrane 506. As with the embodiment of FIG. 4, the tube 502 has a lock section 512, in this instance a button shape, within the inflatable area.

Preferably, the edge 530 of the substrate 520 has a radius of curvature in the range from about 10 to 13, and most preferably 10 to 12 inches. The edge 540 of the substrate member 520 preferably has a radius of curvature in the range from about 10 to 7 inches and most preferably a radius of curvature in the range from about 10 to 8 inches. The radius of curvature required for a proper fit for a male with large fingers is significantly greater than that for a woman with small fingers. Similarly, the radius of curvature required for a proper fit for a woman with large fingers is significantly greater than that for a child with small fingers. The need for the finger cuff to be curved is due to the importance of the cuff to conform to the taper of the user's finger in order to provide the essential firm contact between the bladder and the finger uniformly across the area of the bladder, in particular, the full length of the region of the finger between the proximal and distal joint which is in contact with the bladder member 505. Thus, the cuffs need to be very flexible to conform to the finger without creases or air pockets.

Example of Preferred Dimensions:

The following preferred dimensions and their ranges are applicable to all disclosed embodiments, although reference numbers are being used from a single figure. This is for ease of description and is not intended to be a limitation.

The length of the tube is preferably no less than several inches or more. As the tube provides communication between the cuff and the recording/storage device the length can vary depending on application. In some embodiments, the tube can be extendable through airtight connectors to enable extension at time of application.

The length of the short side “B” of the inflatable region of the membrane 505, as seen in FIG. 5, is preferably in the range from about 0.63 to 1.5 inches.

The width “C” of the substrate member 520 is preferably in the range from about 1⅛ to 1½ inches.

The width “D” between the two long sides of the inflatable region 506 of the membrane 505 is in the range from 0.6 to 1.25 inches and is selected to correlate to the distance between the distal and proximal joints of the middle phalange of a finger, preferably, the middle finger.

The width “E” of the hook area 508 is preferably in the range from ½ to ¾ of an inch.

The loop region 508 of the hook and loop member is preferably larger than the region of the inflatable member.

The radius of curvature of the substrate edge 540 can be in the range from 7 to 13 inches and the radius of curvature of the shorter the long substrate edges can be in the 7 to 10 inches and 10 to 13 inches for the longer of the long substrate edges.

The inflatable member is preferably has an active surface area in the range from 0.9 to 2.5 square inches. Preferably, for a large finger the active surface area is in the range from about 1.5 to 2 square inches. Preferably, for a small finger the active surface area is in the range from about 0.9 to 1.3 square inches.

The distance “R” between the two short sides of the substrate member can be in the range from 3 to 6 inches, and preferably is in the range from 3¾ to 5¼ inches.

Reference to the length of a side of the inflatable region or the substrate member is intended to be inclusive of the distance between two opposing walls of a filleted or chamfered rectangle.

In FIG. 6A the cuff 500 is seen on the middle phalange of the user's middle finger with the tube 502 leading to a signal processing and data recording and storage device 600. From the recording device 600, the data can be sent to any device used to gather and analyze data within the facility. The transfer of data can be through any means known at the time in the computer arts and applicable to the application. Alternatively, the data can be analyzed and read directly from the recording device 600.

The finger has a bone in the center and two arteries, one on each side. The cuff 500 is placed on the user's finger with the flexible bladder in contact with the two arteries. Upon pressurization of the bladder to about 40 mmHg, the cylindrically pressurized cuff squeezes the finger tissue and unloads the finger arteries. This eliminates the elasticity function of the artery with the bladder around the finger then providing the elastic restoring force that is, the bladder becomes the elastic arterial wall. The bladder also now contains the pulse pressure wave. No artery is squeezed against a bone, no circulation is impeded and no palpation method is used. As previously stated, the bladder has been pressurized to below the diastolic pressure. By way of extreme contrast, a brachial artery cuff plethysmograph must be pressurized to a level above the arterial systolic pressure.

This ballooning is familiar through party balloons used by children. If they are pressurized weakly, the balloon doesn't change size, but merely stands up. If they are pressurized beyond this, these balloons hyper-inflate and make what is normally called a balloon. Urethane does hyper-inflate if the pressures are high enough. In a hydrostatic cuff, hyper-inflation must be avoided.

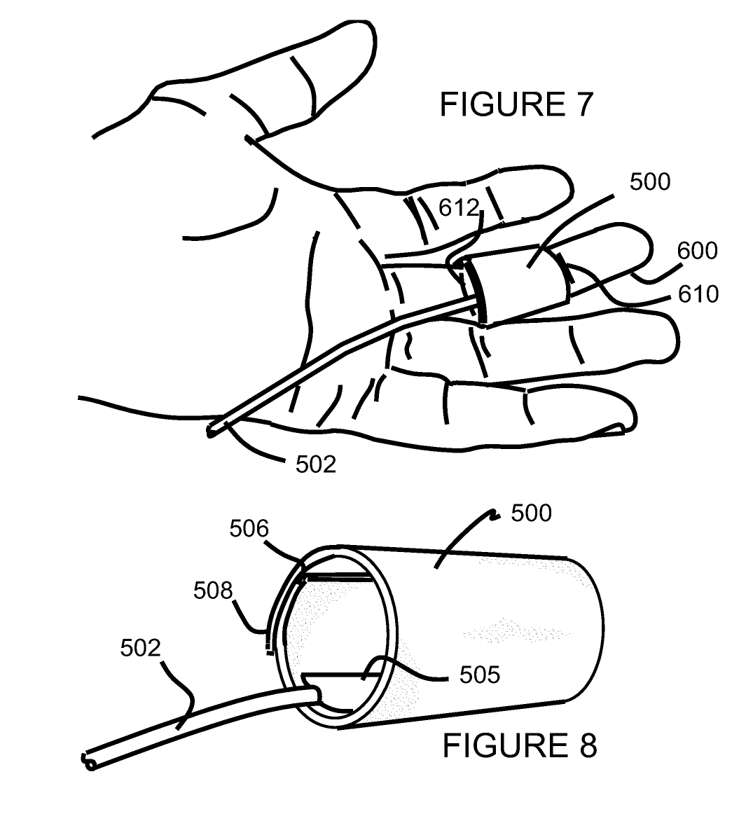

In FIG. 7 the cuff 500 has been wrapped and the conical configuration is clearly illustrated. It is an aspect of the invention to achieve this configuration in order for the inflatable membrane to uniformly contact the region of middle phalange 600 from the distal joint 610 to the proximal joint 612. The tube 502 is seen clearly in this Figure extending from the cuff 500.

At least two failsafe mechanisms are employed to prevent over inflation of the cuff, which would kill the finger in extended use. One is that the software checks the pressure every quarter of a second and if the pressure is high, the whole systems shuts down and the pressure is electromechanically released.

The structure of the present invention has been designed to be an autonomous, self-powered unit. Since, currently available valves require 180 mA at about 4 V to keep a normally open valve closed, the present invention employs a normally closed valve in order to conserve battery power. In this case the excitation initiates a venting action, releasing the pressure in the bladder.

A general power failure would not passively result in the release the pressure in the bladder. Therefore, as a first failsafe step, the absolute pressure is read four times a second and can shut the whole process down using software commands and controls. As stated heretofore, the set point for bladder pressurization is about 50 mmHg, well below diastolic pressure and therefore not considered to be injurious to a finger. It is preferable that control of the set point is via the software; however a maximum set point can be programmed in for additional safety.

A second failsafe is a poppet valve that simply opens when a predetermined maximum pressure is reached. In a conventional automatic sphygmomanometer there is no need for a poppet valve because the cuff is holding back pressure, which can be as high as 200 mmHg or more, and, if the power failed, it would open and release the pressure alleviating any danger to the wearer. The prior art cuff uses a normally open valve and excitation of the electrical version closes the valve. As stated heretofore, the present invention employs a normally closed valve, the opposite of prior art cuffs. The poppet valve used herein is set at about 1 to about 1.2 PSI. Thus, if the pressure increased beyond a predetermined level, the poppet valve would release it passively, without electrical control.

In FIG. 8 the cuff 500 is seen without a user's finger. The tube 502 extends from the membrane 505 and the hook 508 has been affixed to the loop 506.

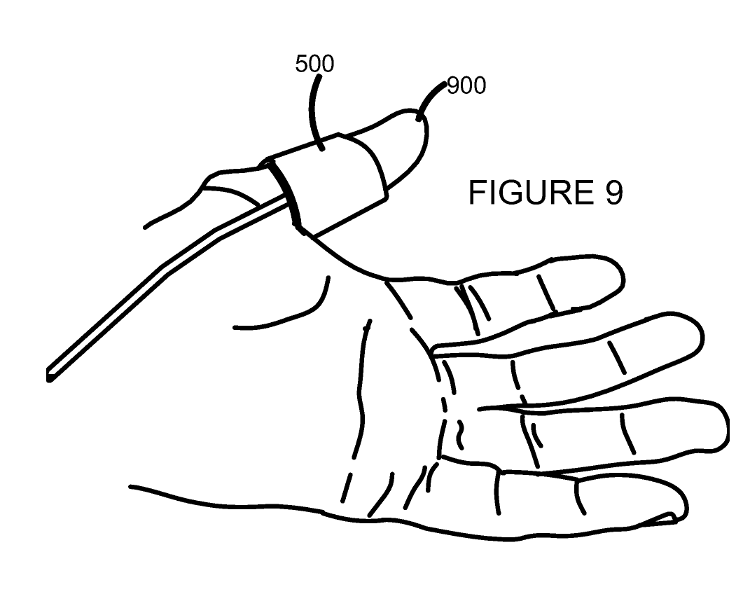

In FIG. 9 the cuff 500 has been placed on the first phalange of the user's thumb 900. The use of the thumb provides advantages as stated heretofore, but nevertheless, other fingers can be used to obtain close to obtain useful readings.

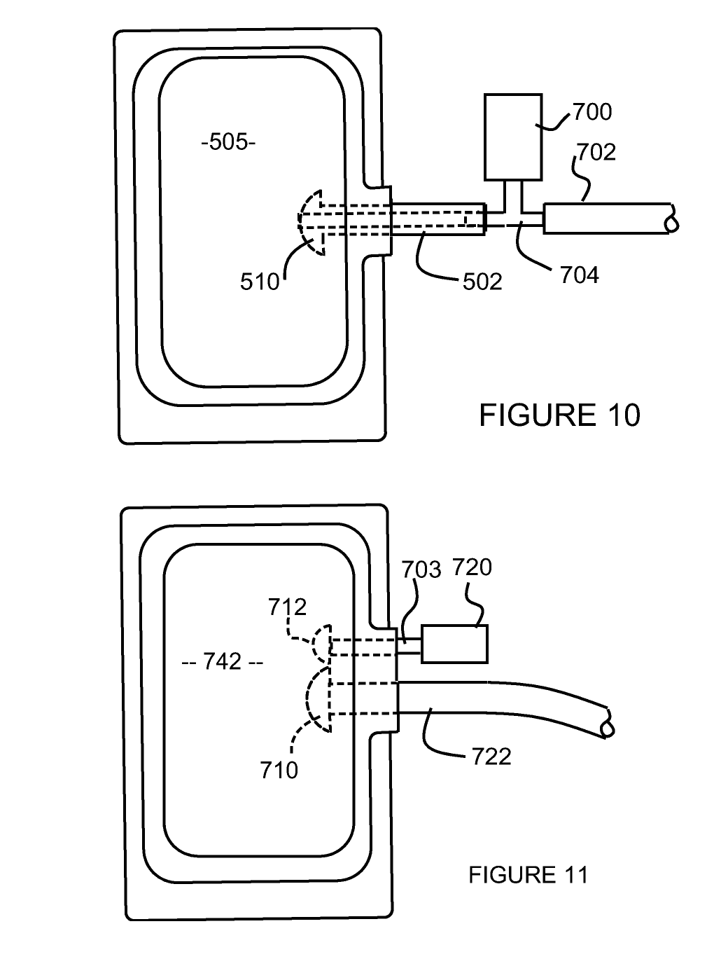

In FIG. 10 the poppet valve 700 is affixed to a manifold which is illustrated as a T-connector 704 that is inserted between the tubing 502 that leads from the interior of the inflatable region 505 and the tubing 702 that leads to a pump located in the electronic device housing 600. Although a T-connector 704 is used in this embodiment, any other methods for situating the poppet valve 700 along the tubing can be used and will be known to those skilled in the art, as for example, through the use of a “Y” shaped manifold.

In the embodiment illustrated in FIG. 11, as with prior embodiments, the inflatable area 742 contains the tubing 722, and respective lock 710, which leads to the electronic device 600 (not illustrated). Additionally a second tube 703 extends into the inflatable area 742 where it is equipped with lock 712. The opposing end of the tube 703 leads to a poppet valve 720. It is noted that more safe guards translate into lower insurance premiums and thus lower costs. As previously noted the tubes 703 and 722 are preferably fused to the urethane inflatable member and the urethane substrate member.

A poppet valve can contain a stainless steel spring and ball device mounted inside a stainless steel tube with barbs on the outside of the stainless tube made to grip the inside of a polymeric tube, such as 722 and/or 703. The valve assembly preferably has a precision valve seat to prevent leakage. A poppet valve can additionally, or alternatively, be included within the electronic device housing 600.

In another alternative a polymeric part, single use valve can be provided in association with the cuff. In the event of over pressuring the bladder, for some unexplained or accidental reason, a polymeric membrane would fail and release the pressure, rendering the cuff unusable at this point. The finger cuffs are reusable, but for health reason can be disposable, in the sense that they are intended to be used only by a single user, such as a single patient in a hospital. The user might use the cuff for an extensive period of time and might remove the cuff temporally, as for example, when bathing or washing hands, but this is considered a single use. Additionally, a user might dispose of a finger cuff if it became soiled, and would then use a new cuff.

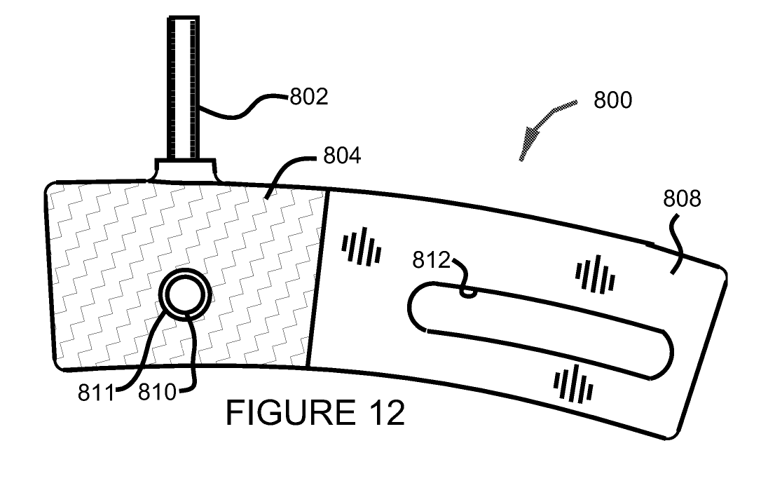

In the embodiment illustrated in FIG. 12 the pressure relief membrane 810 can be formed directly on the bladder (not show), though the contact between the frangible membrane 810 and the finger can affect adversely affect the pressure which is required to burst the membrane. Preferably, the frangible membrane 810 is formed on the substrate member 808 and an opening 811 is provided in the loop material 804. Bursting of the membrane 810 will cause air to be released between the hook and the loop 804 sections of the cuff, since the hook and loop 804 system does not form an air tight seal. However, preferably an elongated slot 812 can be provided in the substrate 808 in the region where the substrate member overlies the membrane 810 when the cuff 800 is in use. This enables the pressure to be released more rapidly in the event of membrane rupture. In another embodiment, the hook section of the fastener can be provided with a plurality of air passage holes which serve the same function as the single elongated opening 812.

Although commonly manufactured in disc form, the rupture membranes also are manufactured as rectangular panels (rupture panels or vent panels). Device sizes preferably range under ¼ inch and can be constructed from various materials, in particular, polymeric films that can rupture at a pressure under two psi.

Alternatively, the frangible membrane can be mounted in the manner of the poppet valve 700 of FIG. 10, or poppet valve 720 of FIG. 11.

### 2—Typical Pressures in the Body

| | kPa | mmHg |
|---|---|---|
| Arterial Blood Pressure | | |
| Maximum (systole) | 13-18 | 100-140 |
| Minimum (diastole) | 8-12 | 60-90 |
| Venous blood pressure | 0.4-0.9 | 3-7 |
| Capillary blood pressure | | |
| Arterial end | 4 | 30 |
| Venous end | 1.3 | 10 |

1 Pa = 145 × 10⁻⁶ psi
1 psi = 6.89 kPa
1 kPa = 1000 Pa = 145 × 10⁻³ psi.
40 mmHg = 40 × 133.3 Pa = 5.33 kPa

Pulse Decomposition Algorithm

The basic components of the algorithm are 1—a peak finder that identifies heartbeats in the derivative data stream, 2—a differentiator that produces the second derivative of the detected heart beat which is then used to find the inversions corresponding to the locations of the component pulses, 3—a digital integrator, implemented as a Bessel filter, that generates the integrated pulse wave form from the differentiated raw signal stream, and from which relative component pulse amplitudes are determined and 4—a low-pass filter that enables identification of the primary systolic peak. Furthermore the frequency content of the data stream is continuously analyzed in order to calculate signal to noise (S/N) figures of merit that determine whether signal fidelity is sufficiently high to permit peak detection and analysis.

Once the temporal locations of the reflection component pulses and the systolic peak are identified, the T13 interval, the time delay between systolic (P1) and iliac peak (P3), is calculated. The P2P1 ratio is calculated using the amplitudes of the P2 peak and the systolic peak, in the integrated pulse spectrum.

Method of Operation

The system of the present invention operates passively at a low constant coupling pressure, such as 40 mmHg. After being provided a calibrated blood pressure reading, the device tracks blood pressure by analyzing the timing and amplitudes of the primary left ventricular ejection pulse as well as the arterial pulse reflections, at the middle phalange of the middle finger.

The system can provide relative, real-time, beat-to-beat pressure measurement values during magnetic resonance imaging. The system can include a transimpedance amplifier and transducer, Bluetooth Dongle, USB D/A Converter and Cables, INISO optically isolated input adapter, automatic Blood Pressure Calibration Unit, and runs on a computer using an operating system such as Windows XP, Vista, Windows 7, and sends analog signals back to a BIOPAC MP Device or third-party ND convertors. A BIOPAC Systems, Inc., HLT100C module can be used to interface the INISO Optically Isolated Input Adapter to the BIOPAC Systems, Inc. MP150 data acquisition system to provide optimal isolation for improved subject safety.

The finger cuff system can be controlled from and stream data to the software running on a PC computer. Communication can be wireless using, for example, the Bluetooth transmission protocol. In a preferred embodiment, the digital sensor features a miniaturized design based on a piezo-electric sensor, weighs ˜114 grams and runs for about 12-hours on a single battery charge.

Since the device tracks pulse reflections that stem from the central arteries, it can be shown to be capable of tracking central blood pressure. Recent experiments furthermore indicate that the technology is particularly suitable as a hemorrhage detector. This is due to the fact that PDA is particularly adept at tracking pulse pressure, which is a sensitive and specific marker for central hypovolemia.

The device's signal quality is sufficiently high as to enable detailed contour analysis of the radial or digital pulse shape, which is influenced by factors such as systolic and diastolic blood pressure, arterial distensibility and the pressure impedance effects of artery/arteriole interfaces. Specifically, it makes the resolution of the component pulse structure of the radial/digital pulse envelope possible.

Transimpedance Amplifier

A 35 mm piezo element has about 0.02 uF capacitance and the voltage it produces measuring pulse is nominally about 1 volt. At 1 Hz, 1 volt on the capacitance of the piezo element causes about 0.1 μamp to flow. A voltage amplifier with gain=1 should be about equal to a transimpedance amplifier with R_{f}=10 Mohm.

Frequency Regime

The frequency regime of the present invention covers the resting breathing fundamental at the low frequency extreme to the upper frequencies contained in the heartbeat. The passband therefore is about 100 mHz to about 60 Hz.

Current Amplification

A transimpedance amplifier converts current input to voltage output. The piezo element converts tensile stress in the PZT element to displacement of electrical charge, Q. Thus Q=k₁₃° F. Since dQ/dt=k₁₃*dF/dt and dQ/dt=i then the transimpedance amplifier's output voltage is proportional to the time derivative of force applied to the sensing element.

If the pulse waveform spectrum is decomposed into a set of sine waves (the Fourier Transform) then the fundamental definition, dQ/dt=w* cos (Ωt) reveals the obvious fact that the derivative of the set of sine functions falls at 20 dB/decade to zero as w approaches D.C. Thus, if the current representing the movement of charge between shorted electrodes is measured instead of the open circuit voltage between them, D.C. blocking is intrinsic in the measurement and no capacitor is needed.

At 100 mHz, its reactance becomes significant relative to the 100M feedback resistance and the low frequency break causes the effective gain of the circuit to fall away towards D.C.

The capacitance of the piezo electrodes appears in parallel to the effective voltage source in the voltage amplifier model and its reactance, greater than 100K at the highest frequency in our passband, is therefore ignored.

The frequency regime used in the present plethysmograph covers the resting breathing fundamental at the low frequency extreme to the upper frequencies contained in the heartbeat. The passband therefore is about 100 milliHz to about 60 Hz.

Advantages of the Transimpedance Amplifier Circuit.

It gets rid of the very large input capacitance which is required to remove low frequency thermal drift from the measured signal.

The circuit offers a very low impedance to ElectroMagnetic Influence from external sources. The high impedance input line offered by the voltage amplifier circuit is, on the other hand, a very good antenna.

Since the output current from the piezo element represents the time derivative of the signal, it is always centered at about zero volts and maximum gain can be used to set the system Noise Figure without fear of the signal clipping at the power supply rails.

By clamping the voltage across the capacitive elements of the piezo sensor, no back emf develops which retards the motion of charge in the circuit and maximum linearity is obtained.

The parts used in the circuit are minimal in number and small in size.

The pulsations can be seen on the pressure gage as loading begins. The spectral content of the pulsation is of primary concern. That is, in ascultatory (the act of listening for sounds made by internal organs, as the heart and lungs, to aid in the diagnosis of certain disorders), oscillometry (an apparatus for measuring oscillations, especially those of the bloodstream in sphygmometry), all a physician or monitoring party wants to see is a disturbance, in contrast with the structure of the disturbance. The present invention is concerned with the structure of the disturbance, that is, the waveform of the disturbance. This is essentially a spectrographic analysis in that it decomposes a pulse wave into its constituent elements, and the constituent elements of the waveform are used to derive data that can be used to support a diagnosis.

In contrast with palpation systems, it would be pointless to press a bladder into arteries in the finger because they are so small and the bladder is relatively large, rounded, and soft. Even pushing a pointed object into a finger artery would be almost pointless because the arteries are so small and, although there is a bone, it would be hard to precisely get the artery between the point and the bone. Everything is slippery and, except for the bone, moves around. Unlike the radial artery, it is at best, difficult to trap a finger artery against a bone.

The energy of the pulse stretches the arterial wall like springs. As the pulse moves forward, the walls give back the stored energy to the pulse. There is a continual storage and release of energy to the elastic walls. Ideally, very little energy is lost as the pulse makes its way to the capillaries. The storage and release of energy slows the pulse from about 1500 meters per second as it would be in a steel pipe to around ten meters per second in the artery.

What is claimed is:

1. A hydrostatic finger cuff diagnostic support means for detecting a pulse waveform, comprising,
an elongated substrate member, said elongated substrate member, having a pair of opposing long side edges and a pair of opposing short edges, said pair of opposing long side edges having a radius of curvature in the range from about 7 inches to about 13 inches
an inflatable member mounted on the obverse side of said elongated member, said inflatable member having a pressurizable interior region,
a tube fixed to said inflatable member and in pneumatic communication with said interior region of said inflatable member,
a first of a hook and loop member affixed to said obverse side of said elongated member and positioned proximate a first of said pair of opposing short edges of said elongated substrate member,
said inflatable member being positioned proximate a second of said pair of opposing short edges of said elongated member,
a second of said hook and loop member affixed to the reverse side of said elongated member.

2. The hydrostatic finger cuff of claim 1, wherein at least one of said substrate member, said inflatable member, and said tube is formed of polyurethane.

3. The hydrostatic finger cuff of claim 1, wherein said long side edges have a length in the range from 3 inches about 6 inches.

4. The hydrostatic finger cuff of claim 1, wherein a first of said long side edges has a radius of curvature in the range from 10 inches about 13 inches and a second of said long side edges has a radius of curvature in the range from 10 inches about 7 inches.

5. The hydrostatic finger cuff of claim 5, wherein a first of said long side edges has a radius of curvature in the range from 10 inches about 12 inches and a second of said long side edges has a radius of curvature in the range from 10 inches about 8 inches.

6. The hydrostatic finger cuff of claim 6, wherein said inflatable member is substantially rectangular and has an active surface area in the range from 0.9 to 2.5 square inches.

7. The hydrostatic finger cuff of claim 1, wherein said inflatable member is substantially rectangular and has an active surface area in the range from about 1.5 to 2 square inches.

8. The hydrostatic finger cuff of claim 1, wherein said inflatable member is substantially rectangular and has an active surface area in the range from about 0.9 to 1.3 square inches.

9. The hydrostatic finger cuff of claim 10, wherein said tube has an inside diameter in the range from 0.05 to 0.075 inches and a first side of said active area of said inflatable member has a length in the range from 0.6 inches to 1.2 inches.

10. The hydrostatic finger cuff of claim 10, wherein a first side of said active area of said inflatable member has a length in the range from 0.63 inches to 0.88 inches and a second side of said active area of said inflatable member has a length in the range from 1 inch to 1.5 inches.

11. The hydrostatic finger cuff of claim 9, wherein a first side of said active area of said inflatable member has a length in the range from 1.25 inches to 0.88 inches and a second side of said active area of said inflatable member has a length in the range from 2.25 inches to 1.5 inches, said inflatable member being inflated to a pressure in the range from 30 mm Hg to a pressure no greater than 60 mm Hg.

12. A hydrostatic finger cuff diagnostic support means for detecting a pulse waveform, comprising,
an elongated substrate member, said elongated substrate member, having a pair of opposing long edges and a pair of opposing short edges, said pair of opposing long edges having a length in the range from 3 inches about 6 inches.
an inflatable member mounted on the obverse side of said elongated member, said inflatable member having a pressurizable active surface area in the range from about 0.9 to 2.5 square inches.
a tube fixed to said inflatable member and in pneumatic communication with said interior of said inflatable member,
a first of a hook and loop member affixed to said obverse side of said elongated member and positioned proximate a first side edge of said elongated substrate member,
said inflatable member being positioned proximate a second side edge of said elongated member,
a second of said hook and loop member affixed to the reverse side of said elongated member.

13. The hydrostatic finger cuff of claim 12, wherein said substrate member and said inflatable member are polyurethane and wherein said tube is polyurethane.

14. The hydrostatic finger cuff of claim 12, wherein said inflatable member is substantially rectangular and has an active surface area in the range from 0.9 to 1.3 square inches.

15. The hydrostatic finger cuff of claim 12, wherein said inflatable member is substantially rectangular and has an active surface area in the range from about 1.5 to 2.25 square inches and wherein a first side of said active area of said inflatable member has a length in the range from 0.6 inches to 1.2 inches.

16. A hydrostatic finger cuff for blood flow property analysis comprising,
an elongated substrate member, said elongated substrate member, have a pair of opposing long edges and a pair of opposing short edges, said pair of opposing long edges having a length in the range from 3 inches about 6 inches.
an inflatable member mounted on the obverse side of said elongated member, said inflatable member having a pressurizable active surface area in the range from about 0.9 to 2.5 square inches.
a tube fixed to said inflatable member and in pneumatic communication with said interior of said inflatable member,
one of a hook and loop member affixed to said obverse side of said elongated member and positioned proximate a first side edge of said elongated substrate member,
said inflatable member being positioned proximate a second side edge of said elongated member,
the other of a hook and loop member affixed to the reverse side of said elongated member,
a pressure relief member, said pressure relief member being in fluid communication with said inflatable member, said pressure relief member being set to open and release pressure at a pressure level of up to 1.2 psi.

17. The hydrostatic finger cuff for blood flow property analysis of claim 16, wherein said tube has a terminal end proximate said inflatable member and a terminal end distal said inflatable member, said pressure relief member being a poppet valve affixed to said tube at a point between said proximal terminal end and said distal terminal end and is set to open and release pressure at a pressure level of up to 1.1 psi.

18. The hydrostatic finger cuff for blood flow property analysis of claim 16, wherein said pressure relief member is a frangible member affixed to said substrate member and in fluid communication with the interior of said inflatable member and is set to open and release pressure at a pressure level of up to 1.1 psi.

19. A diagnostic support method using a finger cuff, said finger cuff comprising:
an elongated substrate member, having an obverse side and a reverse side, an inflatable member mounted on the obverse side of said elongated member, said inflatable member having a pressurizable interior region,
a tube fixed to said inflatable member and in pneumatic communication with said interior of said inflatable member,
a first part of a connector member affixed to the obverse side of said elongated member and positioned proximate a first side edge of said elongated substrate member,
said inflatable member being positioned proximate a second side edge of said elongated member,
a second part of a connector member affixed to the reverse side of said elongated member,
comprising the steps of:
encircling a user's finger with said finger cuff,
affixed said first part of a connector member to said second part of the connector member such that said inflatable member is in firm, circumferential contact with the finger of said user,
inflating said inflatable member to a pressure below the user's diastolic pressure while maintaining said inflatable member in circumferential contact with the middle phalange of the user's finger,
generating pressure fluctuations which correspond to the user's blood pressure.

20. The method of claim 24, further comprising the step of inflating said inflatable member to a pressure in the range from 30 mmHg to no greater than 60 mmHg.

21. The method of claim 24, further comprising the step of inflating said inflatable member to a pressure in the range from 30 mmHg to 50 mmHg.

22. The method of claim 24, further comprising the step of transmitting pressure pulses from said inflatable member to a pressure sensor, converting analog output from said pressure sensor to a digital signal,
transmitting said digital signal to a computer, and
converting said digital signal in said computer to a corresponding blood pressure value.

23. The method of claim 19, where said user's finger is a thumb.
